(19) United States
(12) Patent Application Publication
(10) Pub. No.: US 2012/0130173 A1
(43) Pub. Date: May 24, 2012

(54) CONTROL DEVICE

(75) Inventors: Theodor Lutze, Balgheim (DE); Olaf Hegemann, Tuebingen (DE)

(73) Assignee: Aesculap AG, Tuttlingen (DE)

(21) Appl. No.: 13/304,833

(22) Filed: Nov. 28, 2011

Related U.S. Application Data
(63) Continuation of application No. PCT/EP2010/055400, filed on Apr. 22, 2010.

(30) Foreign Application Priority Data
May 29, 2009 (DE) 10 2009 024 243
Sep. 14, 2009 (DE) 10 2009 042 490

(51) Int. Cl. A61B 1/00

(52) U.S. Cl. 600/146

(57) ABSTRACT

A control device is provided, particularly for use in endoscopes or the like. The control device has proximal and distal end sections which each incorporate an articulation zone and a bending resistant central section arranged therebetween. The control device also comprises outer and inner hollow cylindrical shafts and a control element arranged between the shafts. Force transmitting longitudinal elements extend substantially from the proximal to the distal end section. The longitudinal elements are arranged at angular intervals in the circumferential direction of the control device and are connected together in the regions of their proximal and distal ends in the circumferential direction. The control device further comprises a holding device with the aid of which a part of an articulation zone is fixable, in a bending resistant manner, with respect to the longitudinal direction of the central section or to a functional unit adjoining the proximal or distal end section.

This application is a continuation of international application number PCT/EP2010/055400 filed on Apr. 22, 2010 and claims the benefit of German patent application number 10 2009 024 243.0 filed on May 29, 2009 and German patent number 10 2009 042 490.3 filed on Sep. 14, 2009.

The present disclosure relates to the subject matter disclosed in international application No. PCT/EP2010/055400 of Apr. 22, 2010 and German applications number 10 2009 024 243.0 of May 29, 2009 and number 10 2009 042 490.3 of Sep. 14, 2009, which are incorporated herein by reference in their entirety and for all purposes.

### BACKGROUND OF THE INVENTION

The invention relates to a control device for precision engineering or surgical applications, for use in endoscopes or the like for example.

The invention relates in particular to a control device for instruments for highly precise, minimally invasive, mechanical applications or surgical applications.

Such control devices are known in the state of the art and they have a proximal end section, i.e. one facing the user/surgeon, and a remote or distal end section with each section comprising an articulation zone and they also have a central section which is arranged between the end sections and is frequently resistant to bending. Furthermore, they also comprise an outer hollow cylindrical shaft, an inner hollow cylindrical shaft and also a control element which is arranged between these shafts and has two or more force transmitting longitudinal elements extending substantially from the proximal to the distal end section of the control device. The force transmitting longitudinal elements are arranged substantially uniformly around the periphery of the control device and are connected to one another in the circumferential direction in the vicinity of the proximal and the distal end sections. Tensile and compressive forces with the aid of which a pivotal movement at the proximal end section can be converted into a corresponding pivotal movement at the distal end section can be transmitted by means of the longitudinal elements.

Control devices of this type are known from WO 2005/067785 A1 for example, wherein a multiplicity of force transmitting longitudinal elements in the form of wires or cables are used, these being disposed directly adjacent to one another in the circumferential direction and thereby providing mutual guidance. The outer and the inner hollow cylindrical shafts are available for the guidance of the force transmitting longitudinal elements in the radial direction, this thereby ensuring guidance of the force transmitting longitudinal elements in each direction.

In general, a manually operable gripping part is mounted at the proximal end of the control device although of course its place could be occupied by motor-actuated operating elements, whilst functional components, in particular tools, cameras, lighting elements and the like can be attached at the distal end which is also referred to as the head.

With the aid of instruments containing such a control device in the field of mechanics for example, complicated and difficult to access interiors, such as engines, machines, radiators and the like for example, can be inspected and repaired or even minimally invasive operations of the type discussed above can be effected.

Conventional control devices can be produced with differing maximally permissible bending angles which arise from the structure or the construction of the articulation zones. This results in working areas of differing sizes for the functional components attached at the distal end.

In a whole host of applications, the instruments provided with the control device need to be removed from the working area and then reintroduced, whether this be for reason of replacing, cleaning, maintaining or supplementing the functional components installed at the distal end section. After the instruments have been reinserted into the previous working area, it is desirable for the previous working position to be re-established in as simple a manner as possible.

The object of the present invention is to minimize the expenditure involved in equipping the instruments for different working areas and/or for various sizes of work areas.

### SUMMARY OF THE INVENTION

In conjunction with the control device described hereinabove, the invention proposes a solution for achieving this object wherein the control device comprises a holding device with the aid of which a part of an articulation zone is fixable, in bending resistant manner, with respect to the longitudinal direction of the central section of the control device or a functional unit adjoined to the proximal or distal end section thereof.

With the aid of the holding device in accordance with the invention that is used in the control device, the maximum bending angle of the distal articulation zone can be set in a variable manner in that the articulation zone is shortened at the proximal end-section end whereby there is a resultant construction-dependent reduction of the maximum bending angle at the proximal end-section end. Since the bending angle at the distal end-section end and the articulation zone thereof depends on the bending angle at the proximal end section, the maximum bending angle of the distal end section is also limited thereby.

Consequently, by virtue of the present invention, the maximum bending angle can be set afresh for every newly arising application utilising one and the same control device, or, it can still be varied even during the application so that the size of the working area being viewed by the operating surgeon can be adjusted in a defined manner e.g. when removing pathological structures using an endoscopic technique.

In accordance with a first variant of the control device in accordance with the invention, the holding device comprises a bending resistant sleeve which is displaceable along the longitudinal axis of the central section of the control device.

If the bending resistant sleeve is pushed from the central section in parallel with the longitudinal axis of the control device over a portion of the proximal articulation zone, this will result in a shortening of the articulation zone and thus a reduction of the maximum bending angle.

The displaceable bending resistant sleeve could, in principle, be accommodated in the interior of the control device. It is preferred however for it to be arranged on the outer periphery of the outer shaft because it is more easily accessible and easier to locate here.

In accordance with another variant of the control device in accordance with the invention, the holding device comprises a holding element which is supported on the functional unit of the proximal end section i.e. typically an operating unit, this giving rise to the advantage that the articulation zone can be shortened from the proximal end side so that the control device will remain unchanged over a greater length and can, for example, be pushed into a trocar.

The holding element that is supported on the operating unit preferably incorporates a ring which can be displaced along the outer periphery of the outer shaft and is held fixedly on the operating unit by means of a bar having a linear guidance mechanism.

It is particularly preferred in the control device in accordance with the invention, that the holding device be designed in such a way that it is positionable and preferably also fixable in one or in a plurality of predetermined positions.

Consequently, the maximum bending angle of the control device can be specified ab initio and in particular, it can also be restricted in predefined manner by the predetermined positions of the holding device.

If a holding device which is supported by the operating unit is used, the maximum bending angle can be adapted in a simple manner to any possible newly arising problem even when the control device in accordance with the invention is in use.

In the control device in accordance with the invention, it is preferred that the force transmitting longitudinal elements be laterally spaced from one another. This prevents the occurrence of frictional forces between the longitudinal elements when the control device is being operated and ensures power-conserving operation thereof.

In dependence upon the type of control device, it can be of importance for spacers to be arranged between the force transmitting longitudinal elements so that the longitudinal elements are held in a given position along the length of the control device.

As an alternative thereto, provision may also be made for the force transmitting longitudinal elements to be arranged so that they are at least partially in direct contact with one another along the longitudinal direction of the control device, whereby, in many cases, sufficient lateral guidance or guidance in the peripheral direction will then be provided by this arrangement alone.

It is particularly preferred that the force transmitting longitudinal elements be guided in the radial direction by the outer and the inner shaft so that particularly simple but nevertheless precise guidance of the longitudinal elements is obtained with an accompanying precisely predictable bending movement of the distal end.

In a preferred embodiment of the invention, the control device has a control element which comprises a hollow cylindrical component wherein the cylinder wall thereof is sub-divided at least in the region of a section between the proximal and distal ends into two or more wall segments which form the force transmitting longitudinal elements.

In this connection, the two or more wall segments can be connected firmly together by means of an annular collar at the distal end of the hollow cylindrical component.

Furthermore, the two or more wall segments can be connected firmly together in the vicinity of the proximal end of the hollow cylindrical component.

It is particularly preferred that the hollow cylindrical component be formed integrally. Here, the actions required when assembling the control device are then particularly simple. Moreover, the one piece component can be manufactured in a particularly precise manner in regards to the mutual orientation of the wall segments.

Control devices of this design comprise, in particular, a hollow cylindrical component which is manufactured from a single length of tubing, whereby the partitioning of the cylinder wall into wall segments is preferably effected by means of a laser cutting process.

Furthermore, control devices of this type can be realized with very small outer diameters of approximately 2 mm or less for example, and in particular, of approximately 1.5 mm, but nevertheless the lumen remaining in the interior thereof is sufficiently large as to allow other functions to be implemented. For example, the lumen still suffices for transporting away pieces of tissue from the location of the operation, especially enabling them to be sucked out, or for bringing a source of light and its associated optical system to the point where the operation is being carried out.

Self evidently, it is also possible for the control devices in accordance with the invention to have diameters of any size.

Especially useful materials for producing the control device, and in particular, the control element in the form of the hollow cylindrical component are steel alloys or nitinol.

In a further preferred control device of the present invention, provision is made for at least sections of the force transmitting longitudinal elements to be disposed helically so that the proximal and distal ends thereof are fixed in different angular positions in the circumferential direction. The effect can thereby be achieved that the pivotal movement of the distal end can take place in a plane other than that of the pivotal movement of the proximal end, or else, that the directions of pivoting of the proximal end and the distal end are quasi opposed so that the control device adopts a U-shape. This is the case when the proximal and distal ends of the force transmitting longitudinal elements are fixed in an angular position differing by 180° in the circumferential direction.

The force transmitting longitudinal elements can be constructed in differing ways and, in particular, be in the form of cables or wires.

Moreover, the force transmitting longitudinal elements may have a banana-like cross section.

As already explained before, in a particularly preferred embodiment, the force transmitting longitudinal elements are formed from a hollow cylindrical component wherein the cylinder wall is slit in the axial direction over the greater part thereof, and in particular, over almost the entire length thereof for the purposes of forming the force transmitting longitudinal elements such as by means of a laser cutting process for example. Hereby, the longitudinal elements are formed by segments of the cylinder wall having cross sections which take the form of a circular arc.

Preferably, the cross section of the wall segments is that of a circular arc which corresponds to an arc angle of approximately 20° or more, and in particular, of 30° or more.

The number of wall segments preferably lies within a range of 4 to 16, more preferably, within a range of 6 to 12.

The spacing between the wall segments in the circumferential direction (this corresponds to the slot width) as measured in angular degrees preferably amounts to approximately 2° to 15°, and more preferably to approximately 4° to approximately 8°.

The slot width resulting from the laser cutting process can be increased if necessary so that the remaining strip-like wall segments can be moved towards each other without making contact. Due to the cross sections of the longitudinal elements being in the form of a segment of a circle, the non-contact making state of the longitudinal elements is also retained in the articulation regions even in the event of tensional or compressive loads; this applies in particular when guiding the longitudinal elements in the radial direction between an inner and an outer shaft.

The two end regions of the hollow cylindrical element remain unslotted so that the longitudinal elements remain connected together by the ring collars.

Furthermore, provision may be made in the control device in accordance with the invention for the extent of the proximal articulation zone in the longitudinal direction of the control device to be different from the extent of the distal articulation zone. Such a measure permits differing transmission ratios to be provided so that a relatively large angular movement at the proximal end results in a small angular change at the distal end section or vice versa.

A further substantial aspect of the control device in accordance with the invention also lies in the fact that the ratio of the lengths of the proximal and the distal articulation zones can be varied freely within given limits. Thus, the length ratio can be adjusted as required to provide a corresponding conversion of the pivotal movement of the proximal end section into a pivotal movement of the distal end section and the functional unit held thereon.

In a further preferred control device in accordance with the invention, provision may be made for at least one of the articulation zones to be resilient so that the bent end sections will at least partially automatically restore themselves after the applied force comes to an end.

Preferably, the articulation zones of the outer and/or inner shaft have a plurality of slots extending in the circumferential direction, these being separated from each other by wall areas in the circumferential direction or the axial direction.

Here too, lengths of one piece tubing can be used for the outer and/or inner shaft.

In conjunction with a control element produced from a length of one piece tubing as was already described hereinabove, the result in the simplest case is that of a very thin-walled but nevertheless mechanically load-bearing structure consisting of three telescoped tubes providing the functions of the outer shaft, the control element and the inner shaft, whereby devices such as grippers for example that have been placed by means of the control device can be actuated and positioned without giving rise to cross talk between the movement of the one and the other element. In particular, a gripper for example can be guided and rotated within the control device without the pivotal angle and the position of the control element itself being altered thereby or the gripping function as such being affected. Counter-movements are provoked to an equally minor extent; rotational movements through 360° are unproblematic.

Moreover, these control devices can easily be dismantled, sterilized and then reassembled.

It is preferred that a respective wall section should comprise two or more, and in particular three or more slots which are arranged one after the other in the circumferential direction. In connection therewith, the slots are preferably arranged such that they are equally spaced from each other in the circumferential direction.

In the axial direction, the articulation zones of the preferred control devices have three or more slots which are arranged adjacent to each other, whereby it is preferred that the adjacent slots be mutually displaced in the circumferential direction. The spacings, by which the slots are spaced from one another in the axial direction, can be the same or they may vary, whereby the properties of the articulated joint, in particular the bending radius, can be influenced.

Typically, provision is made for the slots to be slots which penetrate completely through the cylinder wall. Very satisfactory bending properties can also be obtained however, if the slots do not penetrate completely through the wall of the shaft, but end, in particular, before reaching the inner periphery thereof. The wall of the shaft as a whole thus remains closed, this being desirable in some applications, in particular, in the case of the outer shaft.

A preferred geometry for the slots is one in which the wall surfaces bounding the slots are arranged at an acute angle to the radial direction. In connection therewith, the opposed wall surfaces of the same slot are preferably mirror-imaged so that the slot is of greater width at the outer periphery of a shaft than it is at a neighbouring inner periphery.

The slots that are spaced from each other in the axial direction are preferably arranged to overlap in the circumferential direction, although they are mutually displaced so as to result in a regular arrangement of the slots.

In connection therewith, the wall surfaces of the slots can be inclined to the axial direction at an angle which differs from 90° so that the width of the slots at the outer periphery is greater than at the inner periphery of the outer shaft. In consequence, adequately large pivotal angles can be realized even when the slot widths are small without having to increase the number of slots or the articulation region having to be extended over a greater axial length.

These and further advantages of the invention are described in greater detail hereinafter with the aid of the drawings.

### BRIEF DESCRIPTION OF THE DRAWINGS

FIG. 1: the partial Figures a, b and c which depict the fundamental structure of a control device in accordance with the invention consisting of the three elements, the outer shaft, the control element and the inner shaft; partial FIG. 1d which depicts an alternative control element; FIGS. 1e and 1f which depict the cross sections of the control element of FIG. 1d or the control device of FIG. 1;

FIG. 2: a typical pattern of movement of the control device in accordance with the invention;

FIGS. 3A and B: exemplary embodiments for the articulation zones of the control device of FIGS. 1a and 1c;

FIGS. 4A and B: further alternative control elements for the control device in accordance with the invention;

FIG. 5: an overall view of a control device in accordance with the invention; and

FIG. 6: the operational principle of a variant of the control device in accordance with the invention.

### DETAILED DESCRIPTION OF THE DRAWINGS

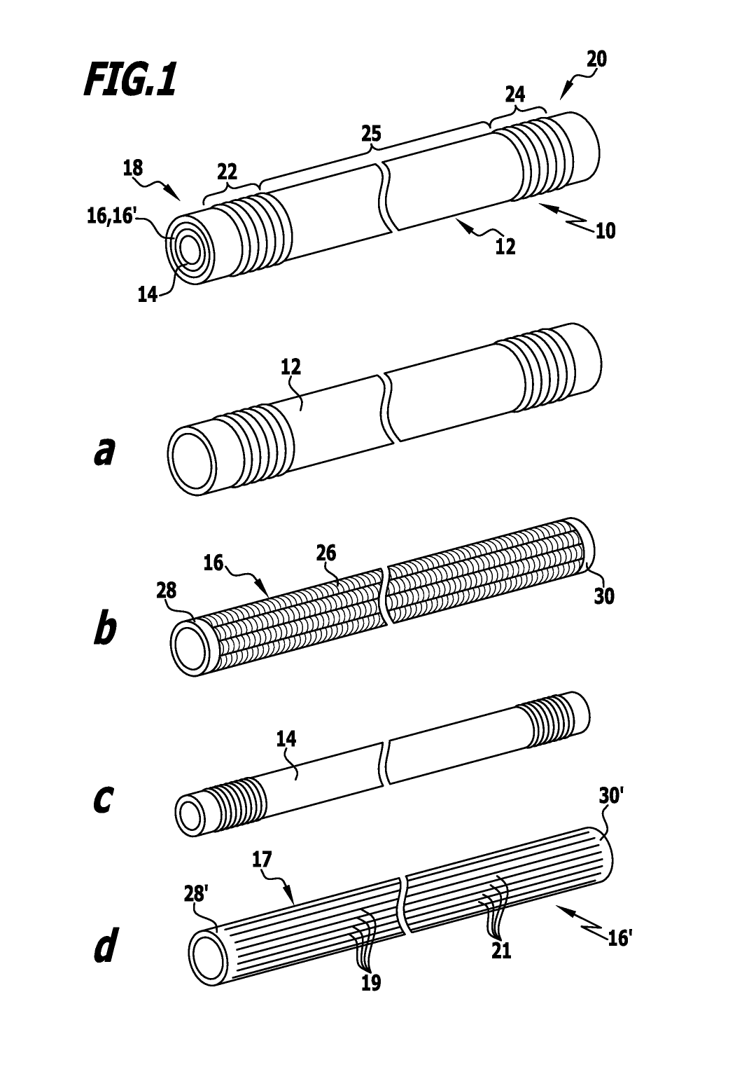

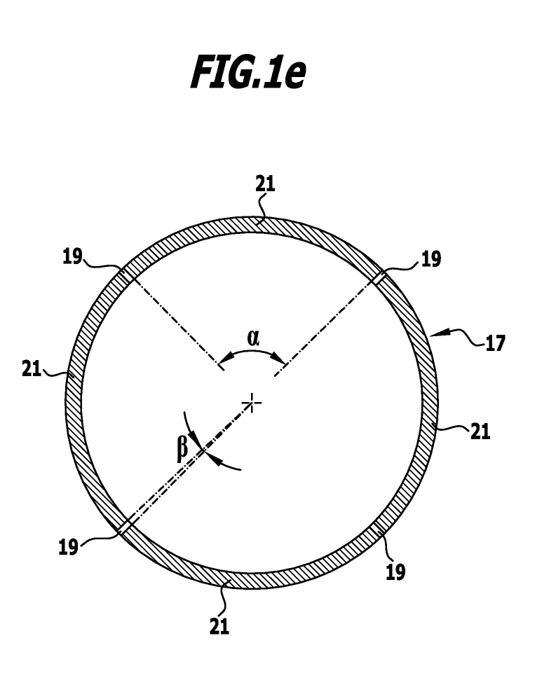

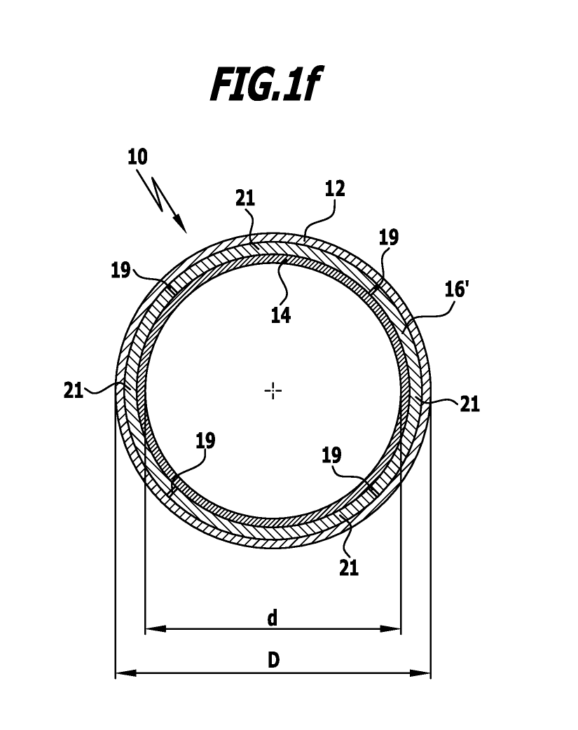

FIG. 1 shows the structure of a control device 10 such as is known from the state of the art, WO 2005/067785 A1 for example and such as can also form the basis for the present invention.

In this connection, the control device 10 comprises an outer hollow cylindrical shaft 12, an inner hollow cylindrical shaft 14 and also a control element 16 which is arranged between these shafts.

The outer and the inner shaft 12, 14 and also the control element 16 are of substantially equal lengths and the external and internal diameters or wall thicknesses thereof are dimensioned in such a way that the control element can be slid into the outer shaft so that it fits precisely and such that the inner shaft 14 fits precisely into the interior of the control element 16. The interior of the inner shaft 14 in the form of a lumen remains free for the introduction of instrument controls, supply lines to a camera or other optical elements and the like. The control element 16 is guided in the radial direction by the walls of the outer and the inner shaft 12, 14.

The control device 10 has a proximal end section 18 and also a distal end section 20 which each comprise a respective articulation zone 22 and 24.

Typically, the articulation zone 22, 24 is formed by an appropriate design of the outer and/or inner shaft 12, 14, whereby numerous proposals are recorded in the state of the art, amongst others, in WO 2005/067785 A1.

In FIG. 1, the articulation zones 21, 24 are indicated merely in the form of bellows type structures.

The individual elements of the control device 10 of FIG. 1 are again illustrated in FIGS. 1a, 1b and 1c, wherein FIG. 1a represents the outer shaft 12, FIG. 1b the control element 16 and FIG. 1c the inner shaft 14.

The structure of the outer shaft 12 in the regions corresponding to the articulation zones 22 and 24 ensures the articulation or bending ability of the outer shaft 12 in this region. As mentioned before for example, bellows-like structures could be used here. Alternatively, the requisite bending properties or flexibility could also be produced by weakening the wall of the outer shaft 12 in the sections corresponding to the articulation zones 22, 24.

The inner shaft 14 in FIG. 1c can exhibit a similar structure to that of the outer shaft 12 in FIG. 1a so that reference can be made to the description of FIG. 1a.

The control element 16 of FIG. 1b comprises a multiplicity of, eight in the present example, force transmitting longitudinal elements which are disposed in parallel with the longitudinal direction of the control element 16 and which are connected laterally to one another at the respective ends of the control element 16 in the circumferential direction such as to form annular collars 28, 30.

FIG. 1d shows an alternative embodiment of a control element 16′ which is produced from a length of one-piece tubing 17 using a laser beam cutting process for example.

The slots 19 formed in the tubing 17 by the laser beam cutting process extend over almost the entire length of the tubing 17 so that, at the proximal and distal end, there only remain non-slotted annular collars 28′, 30′ which interconnect the wall segments 21 functioning as force transmitting longitudinal elements.

FIG. 1e shows a cross section through a control element of FIG. 1d, in which however only four wall segments 21 are present. The circular arc segments of the wall segments 21 correspond to an arc angle α of approximately 82° to 86°. The extent of the slots 19 in the circumferential direction corresponds to an angle β of approximately 4° to 8°.

FIG. 1f shows the cross section of the control device 10 wherein the control element 16′ of FIG. 1d is used as a control element although with a total of four segments 21.

The outer diameter D of approximately 2.5 mm and the interior diameter of approximately 1.8 mm are mentioned by way of example.

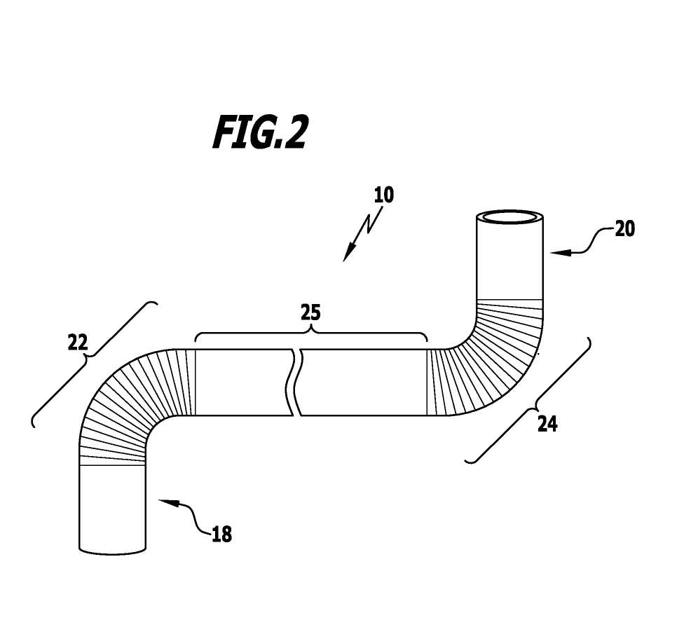

Due to the guidance of the force transmitting longitudinal elements 26 between the outer and the inner shaft 12, 14 in the control device 10, pivoting of the proximal end section 18 results in bending of the articulation zone 24 at the distal end section through the same angular amount and in the same pivotal plane but in the opposite direction. Such a situation is illustrated in FIG. 2.

The design of the articulated sections in the form of the flexible sections 22, 24 of the inner and outer shaft can be of many types.

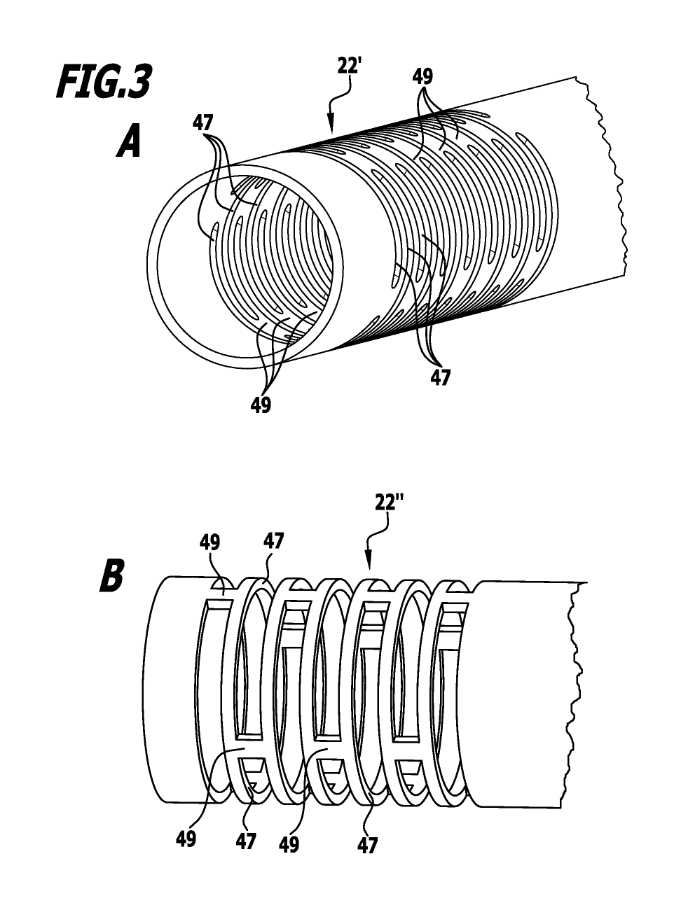

FIGS. 3A and 3B show two variants of related designs for the flexible sections, here in the form of the sections 22′ and 22″ of an outer shaft 12. The same kind of design is also suitable for the flexible sections of the inner shaft 14.

Common to the two variants is the use of a slotted structure in the form of circumferentially extending slots 47 in the hollow cylindrical shaft. Preferably, two or more slots that are separated from each other by webs 49 are provided along a peripheral line. Since an arrangement of slots along just one peripheral line would only allow a very small angle of pivoting, a plurality of axially spaced peripheral lines incorporating slots 47 are provided in typical slotted structures for the articulation zone 22′. It is preferred that adjacent slots 47 in the axial direction be mutually displaced in the circumferential direction thereby giving rise to the possibility of bending in several planes.

There are two slots 47 per peripheral line which are separated from each other by webs 49 in FIG. 3B. In FIG. 3A, there are three slots 47. In both cases, the slotted structure typically consists of a multiplicity of slots 47 which are arranged along several imaginary peripheral lines that are spaced from each other in the axial direction. The permissible angle of pivoting can be predetermined in a very simple manner and also further properties of an articulation section such as the bending resistance for example, can be adapted to the particular application by the choice of the slotted structure and the number of slots.

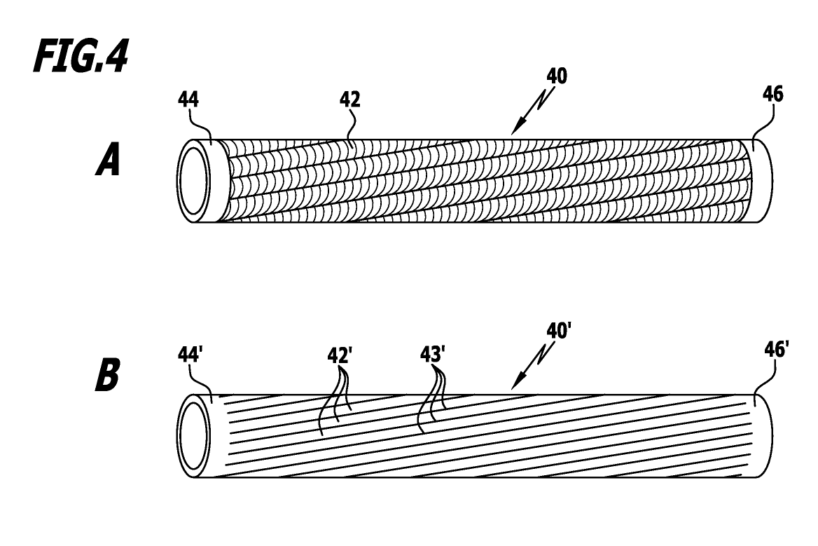

Pivoting of the distal articulation section in other desired predetermined directions with reference to the pivotal movement of the proximal end i.e. even in directions which do not lie in the same plane is possible when using a control element in which the proximal and distal ends of the force transmitting longitudinal elements are set in angular positions in the circumferential direction which differ by a certain angular amount, by 180° for example as shown in the example in FIGS. 4A and 4B.

FIG. 4A shows a control element 40 for the control device 10 in accordance with the invention wherein eight force transmitting longitudinal elements 42 are arranged helically over their entire length and are fixed at the proximal and distal annular collars 44, 46 with a displacement of 180°.

Here, a pivotal movement of the proximal section 18 of the control device 10 in the upward direction likewise leads to an upward pivotal movement of the distal section 20 in the same plane.

In consideration of the fact that the diameters of typical control elements amount to only a few millimetres, but on the other hand the necessary length of the control elements amounts to 10 cm or significantly more, the angles at which the helically arranged longitudinal elements depart from the longitudinal direction of the control elements are substantially smaller than FIGS. 4A and 4B might possibly suggest. In order to clarify this to a greater extent, two numerical examples are presented here:

In the case of an instrument such as is typically employed in neurosurgery, the length of the control device amounts to approximately 30 cm, the length of the associated control element 40, 40′ thus likewise amounts to 30 cm. The outer diameter of the control element 40, 40′ typically amounts to 1.7 mm. If one selects an angular displacement of 180° for the angle at which the proximal and distal ends of the force transmitting longitudinal elements 42, 42′ are fixed at the respective annular collars 44, 46 and 44′, 46′, then this results in a helical pattern for the longitudinal elements wherein the helix is inclined at an angle of approximately 0.5° to the longitudinal axis of the element.

In the case of an instrument that is employed for laparoscopy, the control device has, for example, a length of 22 cm which corresponds to the length of the control elements 40, 40′. The outer diameter of the control element 40, 40′ is comparatively large and amounts to approximately 9.7 mm. For this shorter length of control device 10 but one having a significantly larger diameter at the same time, one obtains an angle of 3.9° at which the helix, along which the force transmitting longitudinal elements 42, 42′ are arranged, is inclined to the longitudinal axis of the control element 40, 40′.

The two examples outlined above should be understood as being extreme examples although for the overwhelming majority of control devices 10 in accordance with the invention, the angle of inclination of the longitudinal elements 42, 42′ with respect to the longitudinal axis of the control element 40, 40′ will fall within the boundaries laid out in these examples.

If one selects a displacement other than those of 180° which were described hereinabove with the aid of FIGS. 4A and 4B, one obtains a differing direction of movement for the distal end 20, for example, in the case of a displacement of 90°, bending of the proximal section 18 in the plane of the paper leads to a perpendicular deflection of the distal end 20 out of the plane of the paper.

Preferably, the control elements for the control devices in accordance with the invention are exchangeable so that different geometries of movement can be imparted to a control device 10 simply by replacing the control element 16, 16′ or 40, 40′.

FIG. 4B shows a variant of a control element 40′ which is formed from a length of one-piece tubing by a laser cutting process in a similar manner to that of the control element 16′ of FIG. 1d. The wall segments 42′ produced thereby are separated from each other by slots 43′ and are only connected together in force-locking manner in the region of the annular collars 44′, 46′. The advantages of the helical pattern of the wall segments are the same as for the control element 40 comprising longitudinal elements 42 running in helical manner.

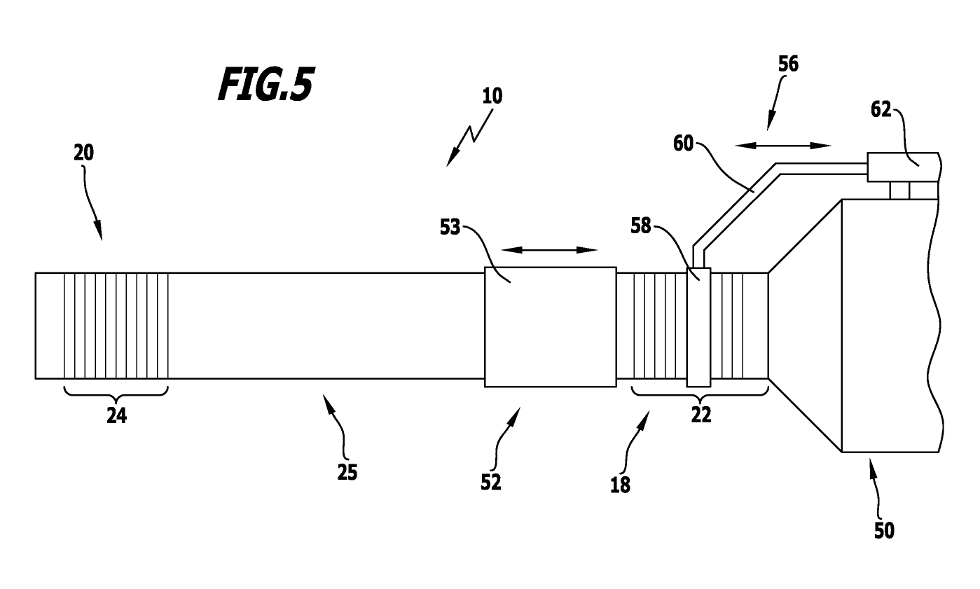

Finally, FIG. 5 shows the present invention on the basis of the control device 10 which is attached to a manipulating device 50 at the proximal end section 18 thereof.

The articulation zones 22 and 24 are of substantially equal length so that when the proximal end section 18 is bent through e.g. 30°, this results in a corresponding bending of the distal end section 20 likewise through 30°. The direction in which the bending of the distal end section 20 is effected is dependent on the choice of control element which is not shown in detail here and upon the fixing of the ends of the force transmitting longitudinal elements such as was described in detail hereinabove.

The control device 10 shown in FIG. 5 additionally has a holding device 52 in the form of a longitudinally displaceable sleeve 53 which is located on the outer shaft of the control device 10 such as to enable it to overlap the central section 25 thereof.

If one displaces the sleeve 53 toward the proximal end section 18 and lets the sleeve 53 overlap this articulation zone 22, then the articulation zone 22 becomes shorter thereby restricting the maximum bending angle thereof. The permissible bending angle in the region of the distal end section 20 can thus be varied so that when removing pathological structures using an endoscopic technique for example a defined working area can be adjusted in the view of an operating surgeon.

FIG. 5 contains an alternative solution for the holding device 52 in the form of the holding device 56 which comprises a longitudinally displaceable ring 58 that is fixed to the manipulating device 50 by means of a doubly cranked bar 60 and a linear guidance mechanism 62. By varying the position of the ring 58 along the section 18, the part of the articulation zone 22 that is available for the bending movement of the proximal end section can be shortened as was described before in connection with the sleeve 53 thereby again permitting only a restricted bending angle at the distal end section 20.

Moreover, it is conceivable in both the case of the sleeve 53 and that of the ring 58 for the arrangement to be fixed in a predetermined position, i.e. with a predetermined overlap of the articulation zone so that the adjusted restricted working area at the distal end section 20 is ensured.

On the other hand, it is also conceivable for the sleeve 53 to be displaced towards the distal articulation section 20 whereby a stepped-up i.e. enhanced pivotal movement then takes place in the region of the distal end section 20 when there is a corresponding pivotal movement of the proximal end section 18.

It is likewise conceivable to provide markings for the position of the sleeve 53 or the ring 58 or its linear guidance mechanism 62 so that once a restriction for the angle has been found it can always be accurately re-established on a later occasion.

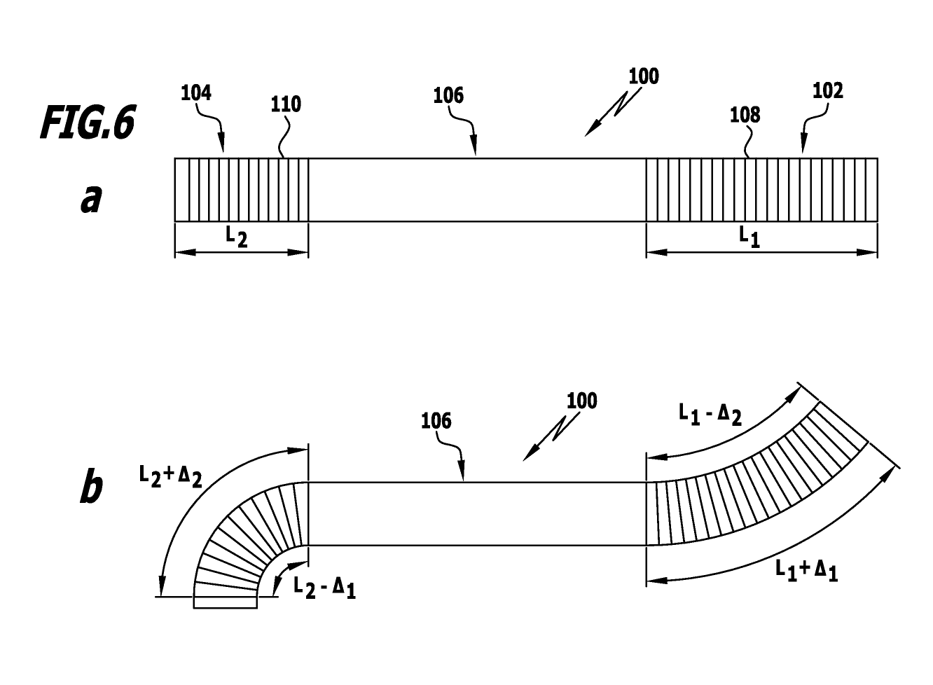

For the purposes of explaining the above described amplifying effect for the pivotal or bending movement at the distal end, attention is drawn to FIG. 6 which shows a control device 100 which has a proximal end section 102, a distal end section 104 and also a central section 106 lying therebetween. Whilst the central section 106 is resistant to bending, the proximal and distal end sections 102, 104 each contain an articulation zone 108 or 110 having a respective length L₁ and L₂ as measured in the axial direction. Hereby, the length L₂ is selected to be shorter than the length L₁. FIG. 6a shows the control device 100 in its basic position in which there are no forces effective on the proximal end section 102.

Should the proximal end region 102 be pivoted out from the axial direction as is made clear in the illustration of FIG. 6b, this then results in the length of the articulation zone 108 being increased to L₁+Δ₁ at the outer radius of the curved end region 102 in the proximal articulation zone 108, whereas it results in a shortened length of L₁−Δ₂ at the inner radius. There are corresponding changes of length for the distal end section 104 with a length at the external radius of L₂+Δ₂ and a length at the inner radius of L₂−Δ₁. Since the lengths L₁ and L₂ of the articulation zones 108, 110 are different, the inevitable result for the distal end section 144 is that of an enhanced bending movement so as to enable it to follow the variations in length prescribed by the proximal end section.

This effect can, for example, also be used in a restricted proximal working area with proportionately small pivotal movements in order to enable full use to be made of the given pivot radius at the distal end and to make as large a working area as possible available at the distal end.

This principle can be used in a variable manner with the present invention, in that the length of one articulation zone in proportion to the other one is varied by a holding device (c.f. FIG. 5).

1. A control device, in particular for use in endoscopes or the like, comprising a proximal and a distal end section each of which incorporates an articulation zone and also a bending resistant central section that is arranged therebetween,
and also comprising an outer hollow cylindrical shaft, an inner hollow cylindrical shaft as well as a control element which is arranged between these shafts and has two or more force transmitting longitudinal elements extending substantially from the proximal to the distal end section of the control device,
wherein the longitudinal elements are arranged at substantially regular angular intervals in the circumferential direction of the control device and are connected to one another in the circumferential direction in the vicinity of the proximal and the distal end sections thereof, wherein the control device comprises a holding device with which a part of an articulation zone is fixable, in bending resistant manner, with respect to the longitudinal direction of the central section of the control device or to a functional unit adjoining the proximal or distal end section thereof.

2. A control device in accordance with claim 1, wherein the holding device comprises a bending resistant sleeve which is displaceable along the longitudinal axis of the central section of the control device.

3. A control device in accordance with claim 2, wherein the bending resistant sleeve is arranged on the outer periphery of the outer shaft.

4. A control device in accordance with claim 1, wherein the holding device comprises a holding element which is supported on the functional unit.

5. A control device in accordance with claim 1, wherein the holding device is positionable and preferably fixable in one or more predetermined positions.

6. A control device in accordance with claim 1, wherein the force transmitting longitudinal elements are arranged so that they are mutually spaced laterally.

7. A control device in accordance with claim 6, wherein spacers are arranged between the force transmitting longitudinal elements.

8. A control device in accordance with claim 1, wherein the force transmitting longitudinal elements are arranged to be at least partially in direct contact with one another along the longitudinal direction of the control device.

9. A control device in accordance with claim 1, wherein the force transmitting longitudinal elements are guided in the radial direction by the outer and the inner shaft.

10. A control device in accordance with claim 1, wherein the control element comprises a hollow cylindrical component having a cylinder wall which is sub-divided at least in the region of a section between the proximal and distal ends into two or more wall segments which form the force transmitting longitudinal elements.

11. A control device in accordance with claim 10, wherein the two or more wall segments are connected firmly together by means of an annular collar at the distal end of the hollow cylindrical component.

12. A control device in accordance with claim 10, wherein the two or more wall segments are connected firmly together in the vicinity of the proximal end of the hollow cylindrical component.

13. A control device in accordance with claim 10, wherein the hollow cylindrical component is formed in one piece manner.

14. A control device in accordance with claim 13, wherein the hollow cylindrical component is manufactured from a single length of tubing, wherein the sub-division of the cylinder wall into wall segments is preferably effected by means of a laser cutting process.

15. A control device in accordance with claim 10, wherein the hollow cylindrical component is made of a steel alloy or nitinol.

16. A control device in accordance with claim 1, wherein at least sections of the force transmitting longitudinal elements are disposed helically so that the proximal and distal ends thereof are fixed in different angular positions as seen in the circumferential direction.

17. A control device in accordance with claim 1, wherein the force transmitting longitudinal elements are in the form of cables or wires.

18. A control device in accordance with claim 1, wherein the force transmitting longitudinal elements have a banana-like cross section.

19. A control device in accordance with claim 18, wherein the extent of the proximal articulation zone in the longitudinal direction of the control device is different from the extent of the distal articulation zone.

20. A control device in accordance with claim 1, wherein at least one of the articulation zones is resilient.

21. A control device in accordance with claim 1, wherein the articulation zone(s) of the outer and/or inner shaft comprise a wall section in which there are a plurality of mutually-spaced slots that extend in the circumferential direction.

22. A control device in accordance with claim 21, wherein two or more, and in particular three or more slots are arranged one after the other in the circumferential direction.

23. A control device in accordance with claim 21, wherein three or more slots are arranged adjacent to each other in the axial direction.

24. A control device in accordance with claim 23, wherein the mutually adjacent slots are mutually displaced in the circumferential direction.

25. A control device in accordance with claim 21, wherein the slots are slots which completely penetrate through the cylinder wall.

26. A control device in accordance with claim 21, wherein the wall surfaces delimiting the slots are disposed at an acute angle to the radial direction.

27. A control device in accordance with claim 26, wherein the opposing wall surfaces of the same slot are mirror-imaged so that there is a larger slot width at the outer periphery of a shaft than there is adjacent to the inner periphery.
